(19) United States
(12) Patent Application Publication
(10) Pub. No.: US 2025/0368601 A1
(43) Pub. Date: Dec. 4, 2025

(54) PROCESS FOR THE PREPARATION OF LISDEXAMFETAMINE

(71) Applicant: Veranova, L.P., Devens, MA (US)

(72) Inventors: Daniel COUGHLIN, West Deptford, NJ (US); Michael MERCADANTE, Devens, MA (US); Cale WEATHERLY, West Deptford, NJ (US); Eugene GUTMAN, West Deptford, NJ (US)

(21) Appl. No.: 19/125,151

(22) PCT Filed: Oct. 27, 2023
(86) PCT No.: PCT/IB2023/060864

§ 371 Date: Apr. 28, 2025

Related U.S. Application Data
(60) Provisional application No. 63/381,374, filed on Oct. 28, 2022.

(51) Int. Cl. C07C 231/14; C07C 231/24

(52) U.S. Cl. CPC C07C 231/14; C07C 231/24

(57) ABSTRACT

A process for the preparation of L-lysine-D-amphetamine dimesylate.

The present invention relates to an improved, efficient and economical process for preparing L-lysine-D-amphetamine dimesylate (also referred to as lisdexamphetamine dimesylate).

### BACKGROUND

L-lysine-D-amphetamine dimesylate (also called lisdexamphetamine dimesylate) is used to treat attention deficit hyperactivity disorder (ADHD) and attention deficit disorder (ADD) in adults and children 6 years of age and older. This pharmaceutical API is also used to treat moderate to severe binge eating disorder (BED). It belongs to the group of medicines called central nervous system (CNS) stimulants. D-amphetamine is the active metabolite of the prodrug lisdexamphetamine dimesylate. The D-amphetamine is liberated from lisdexamphetamine enzymatically following contact with red blood cells. The conversion is rate-limited by the enzyme, which prevents high blood concentrations of D-amphetamine and reduces the abuse potential of lisdexamphetamine at clinical doses. L-lysine-D-amphetamine dimesylate has the IUPAC name of (2S)-2,6-Diamino-N-[(1S)-1-methyl-2-phenylethyl]hexanamide dimesylate and has the chemical structure:

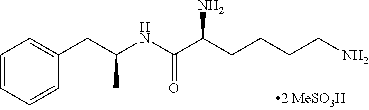 [SMILES: C[C@@H](Cc1ccccc1)NC(=O)[C@@H](N)CCCCN]

U.S. Pat. Nos. 7,105,486; 7,659,253, and 10,927,068 disclose methods for preparing L-lysine-D-amphetamine dimesylate from N,N′-bis-Boc-L-Lys(Boc)-OSu and D-amphetamine free base. For example, U.S. Pat. No. 10,927,068 discloses and claims using alkyl tetrahydrofuran as a solvent for preparing L-lysine-D-amphetamine dimesylate from N,N′-bis-Boc-L-Lys(Boc)-OSu. The invented process is more economical since N,N′-bis-Boc-L-Lys(Boc)-OSu is commercially available and the invented process further avoids the use of an alkyl tetrahydrofuran as a solvent for the pharmaceutical manufacture of L-lysine-D-amphetamine dimesylate and allows an efficient replacement of the reaction solvent with another solvent during the process.

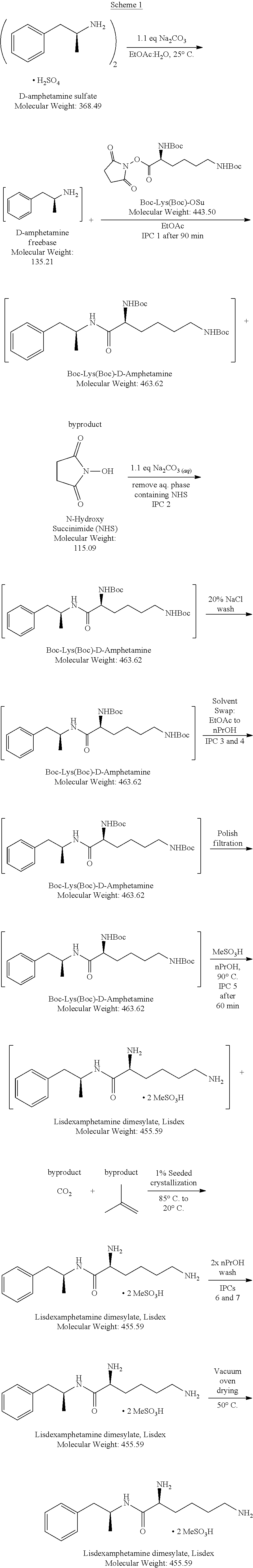

The invented process summarized in Scheme 1 is a more efficient, continuous and convergent manufacturing process that avoids a Class 3 solvent for manufacture of L-lysine-D-amphetamine dimesylate.

### DETAILED DESCRIPTION OF THE INVENTION

### Definitions

The term “about” or “approximately” means an acceptable error for a particular value as determined by a person of ordinary skill in the art, which depends in part on how the value is measured or determined. In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, or 0.5% of a given value or range.

The term “ambient temperature” means one or more room temperatures between about 15° C. to about 30° C., such as about 15° C. to about 25° C.

The term “consisting” is closed and excludes additional, unrecited elements or method steps in the claimed invention.

The term “consisting essentially of” is semi-closed and occupies a middle ground between “consisting” and “comprising”. “Consisting essentially of” does not exclude additional, unrecited elements or method steps which do not materially affect the essential characteristic(s) of the claimed invention.

The term “comprising” is inclusive or open-ended and does not exclude additional, unrecited elements or method steps in the claimed invention. The term is synonymous with “including but not limited to”. The term “comprising” encompasses three alternatives, namely (i) “comprising”, (ii) “consisting”, and (iii) “consisting essentially of”.

The group “Boc” refers to the tert-butyloxycarbonyl protecting group used to protect amine groups in organic synthesis. The term API refers to active pharmaceutical ingredient, which in this invention is L-lysine-D-amphetamine free base or L-lysine-D-amphetamine dimesylate.

The present disclosure provides an efficient, convergent and continuous process to prepare L-lysine-D-amphetamine dimesylate (lisdexamphetamine dimesylate) from D-amphetamine sulfate. The protected intermediate formed during the synthesis process in a biphasic solvent mixture ethyl acetate and water is purified by treatment with sodium carbonate, aqueous sodium chloride, solvent replacement of ethyl acetate with n-propanol and polish filtration prior to addition of methane sulfonic acid (thus, eliminating the need for expensive, industrial chromatography and isolation of a problematic intermediate), dried by azeotropic distillation. No isolation is carried out until a crystalline lisdexamphetamine dimesylate wet cake is obtained by addition of a seed crystal, followed by washing with n-propanol and drying under vacuum with heating. The crystalline lisdexamphetamine dimesylate produced is a pure API obtained in high yield. The process described herein has industrial applicability, with further improvements to yield (e.g., minimal isolations) and safety (e.g., elimination of Class 3 solvents such as alkyl tetrahydrofurans).

The continuous process, as summarized in Scheme 1, comprises (a) converting a D-amphetamine sulfate to D-amphetamine as a free base; (b) adding D-amphetamine to a biphasic reaction mixture comprising ethyl acetate, water and N,N′-bis-Boc-L-lysine succinimide or N,N′-bis-Boc-L-lysine(boc)-OSu to form an intermediate compound, the intermediate compound being N,N′-bis-Boc-L-lysine-D-amphetamine; then phase extracting the N,N′-bis-Boc-L-lysine-D-amphetamine from the biphasic reaction mixture after step (b) with one or more aqueous solutions (Na₂CO₃, NaCl), solvent replacement of ethyl acetate with n-propanol, polish filtration to remove to remove starting reactants and reaction by-products, thereby forming a purified mixture comprising the intermediate compound N,N′-bis-Boc-L-lysine-D-amphetamine; and finally addition of methane sulfonic acid to form L-lysine-D-amphetamine dimesylate, which is crystallized using seed crystallization to afford crystalline L-lysine-D-amphetamine dimesylate.

According to one embodiment of the invention, a continuous process is disclosed for preparing L-lysine-D-amphetamine dimesylate, comprising the steps of: (a) reacting D-amphetamine free base, having the structure

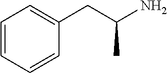

with at least about two equivalents of N,N′-bis-Boc-L-Lys(Boc)-OSu, having the structure

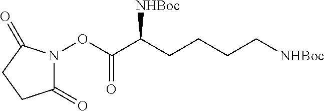 [SMILES: CC(C)(C)OC(=O)NCCCC[C@H](NC(=O)OC(C)(C)C)C(=O)ON1C(=O)CCC1=O]

in a biphasic mixture of ethyl acetate and water, heating the mixture from 25° to 35° C. with stirring to form N,N′-bis-Boc-L-lysine(Boc)-D-amphetamine, having the structure

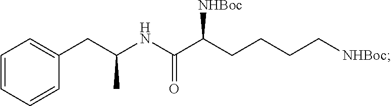 [SMILES: C[C@@H](Cc1ccccc1)NC(=O)[C@H](CCCCNC(=O)OC(C)(C)C)NC(=O)OC(C)(C)C]

and
(b) adding about 1 equivalent of sodium bicarbonate, then separating and removing the aqueous layer of the biphasic mixture and the replacing ethyl acetate portion of the biphasic mixture that includes the N,N′-bis-Boc-L-lysine(Boc)-D-amphetamine with n-propanol as a solvent, then adding at least about two to about ten equivalents of methane sulfonic acid with heating up to 90° C. and stirring to result in the product L-lysine-D-amphetamine dimesylate.

In a separate embodiment, in step (a), D-Amphetamine free base is reacted with about two equivalents of N,N′-bis-Boc-Lys(Boc)-OSu in a biphasic mixture of ethyl acetate and water. The reaction of step (a) is stirred from 1-2 hours, heating the mixture from 25° to 35° C. In step (b), N,N′-bis-Boc-L-lysine-D-amphetamine is reacted with about two to about 10 equivalents of methanesulfonic acid in n-propanol.

In an exemplary embodiment, the solvents used in accordance with the invention a biphasic mixture of water with an organic solvent having limited water solubility. Suitable solvents include ethyl acetate, butyl acetate, benzene, n-butanol, n-propanol, cyclohexane, 1,2-dichloroethane, dichloromethane, ethyl acetate, di-ethyl ether, heptane, hexane, methyl-t-butyl ether, methyl ethyl ketone, pentane, di-iso-propyl ether, toluene, and xylene. In specific embodiments, the solvent used in step (a) is ethyl acetate and water and in step (b) ethyl acetate is replaced with n-propanol.

The amount of solvent added to the reaction mixture depends on the weight of the reactants. In general, the weight ratio of the solvent to the reactants in step (a) may range from about 2:1 to about 70:1. In some embodiments, the weight ratio of the solvent to the reactants in step (a) may range from about 3:1 to about 50:1, from about 4:1 to about 20:1, from about 5:1 to about 15:1, or from about 6:1 to about 10:1.

In another embodiment, there is provided a continuous process for preparing L-lysine-D-amphetamine dimesylate comprising the steps of: (a) reacting D-amphetamine free base having the structure

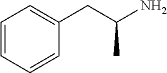

with about two equivalents of N,N′-bis-Boc-Lys(Boc)-OSu having the structure

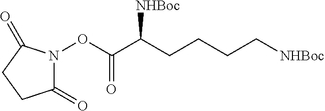 [SMILES: CC(C)(C)OC(=O)NCCCC[C@H](NC(=O)OC(C)(C)C)C(=O)ON1C(=O)CCC1=O]

in a biphasic mixture of ethyl acetate and water, heating the mixture from 25° to 35° C. for 1-2 hours with stirring to form N,N′-bis-Boc-L-lysine(Boc)-D-amphetamine having the structure

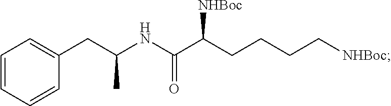 [SMILES: C[C@@H](Cc1ccccc1)NC(=O)[C@H](CCCCNC(=O)OC(C)(C)C)NC(=O)OC(C)(C)C]

(b) adding about 1 equivalent of sodium bicarbonate and stirring the biphasic mixture up to 1 hour, maintaining a temperature for the stirred mixture of from 20° to 25° C., then separating and removing the aqueous layer of the biphasic mixture;
(c) after removing the aqueous layer of the biphasic mixture in step (b), the ethyl acetate portion of the biphasic mixture that includes the N,N′-bis-Boc-L-lysine(Boc)-D-amphetamine is further washed with an aqueous solution of sodium chloride and the biphasic mixture formed again is stirred up to 1 hour, maintaining a temperature for the stirred mixture of from 20° to 25° C., then the aqueous layer of the biphasic mixture formed is separated and removed;
(d) after removing the aqueous layer of the biphasic mixture in step (c), replacing ethyl acetate portion of the biphasic mixture that includes the N,N′-bis-Boc-L-lysine(Boc)-D-amphetamine by concentrating the ethyl acetate under vacuum distillation and heating to 60° C. and adding n-propanol as a replacement solvent that includes the N,N′-bis-Boc-L-lysine(Boc)-D-amphetamine;
(e) polish filtering the N,N′-bis-Boc-L-lysine(Boc)-D-amphetamine in n-propanol solvent from step (d) to remove any remaining inorganic salts;
(f) adding from about two to ten equivalents of methane sulfonic acid to the N,N′-bis-Boc-L-lysine(Boc)-D-amphetamine in n-propanol solvent from step (e), heating the mixture from 85° to 90° C. with stirring for 1-2 hours to result in the product L-lysine-D-amphetamine dimesylate;
(g) cooling the L-lysine-D-amphetamine dimesylate product from step (f) and adding a 1 percent by weight of crystalline L-lysine-D-amphetamine dimesylate as a seed to crystallize the L-lysine-D-amphetamine dimesylate product; and
(h) washing the crystallized L-lysine-D-amphetamine dimesylate product with at least one portion of n-propanol solvent, followed by drying the crystallized L-lysine-D-amphetamine dimesylate product under vacuum at 50° C.

In some embodiments, the L-lysine-D-amphetamine dimesylate product is prepared from an amphetamine salt in the continuous process. In one embodiment the amphetamine salt is D-amphetamine sulfate. Non-limiting examples of suitable amphetamine salts include amphetamine bitartrate, amphetamine sulfate, amphetamine aspartate, amphetamine saccharate, amphetamine hydrochloride, and amphetamine phosphate. In a separate embodiment, the L-lysine-D-amphetamine dimesylate product is prepared from an amphetamine salt that is first converted to the free base by contact with a base, followed by extraction with the solvent used in the process. Suitable bases include, without limit, hydroxides of alkali metals and alkaline earth metals such as, sodium hydroxide, sodium carbonate, potassium carbonate or potassium hydroxide. In an exemplary embodiment, D-amphetamine sulfate is converted to D-amphetamine free base using sodium carbonate.

In some embodiments, N,N′-bis-Boc-L-lysine(Boc)-D-amphetamine in n-propanol solvent is reacted with about two to about ten equivalents of a pharmaceutically acceptable acid and heating the mixture from 85° to 90° C. with stirring for 1-2 hours to result in the product L-lysine-D-amphetamine as an acid addition salt. In one embodiment, the acid is methanesulfonic acid and API produced is the product L-lysine-D-amphetamine mesylate. Contact with the acid removes the Boc protecting group(s) from the N,N′-bis-Boc-L-lysine(Boc)-D-amphetamine and forms an acid salt of the compound. Non-limiting examples of suitable acids include methanesulfonic acid, hydrochloric acid, oxalic acid. In specific embodiments, the acid may be methanesulfonic acid.

The amount of methanesulfonic acid added to the purified mixture of N,N′-bis-Boc-L-lysine(Boc)-D-amphetamine may range from about 1:1 to about 10:1. In some embodiments, the molar ratio of the acid to N,N′-bis-Boc-L-lysine(Boc)-D-amphetamine may range from about 2:1 to about 10:1, or from about 2:1 to about 6:1.

Contact with the acid is generally conducted at a temperature ranging from about 40° C. to about 100° C. In some embodiments, the temperature may range from about 60° C. to about 90° C., from about 80° C. to about 90° C., or from about 85° C. to about 90° C. The duration of contact with acid at the elevated temperature may proceed for at least about 2 hours, at least about 4 hours, at least about 8 hours, at least about 12 hours, or at least about 24 hours.

The resulting API L-lysine-D-amphetamine as an acid addition salt may then be cooled to ambient or room temperature. In one embodiment, the API L-lysine-D-amphetamine mesylate is seeded with a crystal of (up to 1% by weight) to crystallize L-lysine-D-amphetamine mesylate. The crystalline wet cake is isolated from the solvent/acid mixture by filtration, centrifugation, or other suitable means. The filtration may be accomplished using a Büchner funnel, a filter funnel, or other filter aid. The filtration may be gravity filtration or vacuum filtration. The filtered product may be washed with the n-propanol solvent used in the process. The final product may be dried at a temperature ranging from about 50° C. to about 80° C. for about 0.5 to about 12 hours in a vacuum oven or a drying oven.

Embodiments and/or optional features of the invention have been described above. Any aspect of the invention may be combined with any other aspect of the invention, unless the context demands otherwise. Any of the embodiments or optional features of any aspect may be combined, singly or in combination, with any aspect of the invention, unless the context demands otherwise.

The invention will now be described further by reference to the following examples, which are intended to illustrate but not limit, the scope of the invention.

### EXAMPLES

### Abbreviations

API active pharmaceutical ingredient
EtOAc Ethyl acetate
n-PrOH n-Propanol (1-propanol)
RT ambient temperature

### Example 1

### D-Amphetamine Free Base from D-Amphetamine Sulfate

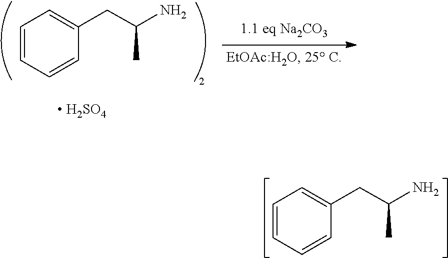

D-Amphetamine sulfate (100.0 g, 1.0 eq.) and ethyl acetate (500 mL, 5 vol) were charged to a clean 3 L glass jacketed reactor (Vessel 1) equipped with overhead agitation, temperature probe, nitrogen inlet, and addition funnel to give a white slurry. Aqueous sodium carbonate (31.64 g, 1.1 eq. in 150 mL, 1.5 vol water) was charged to the reactor over 15 min. using an addition funnel, while maintaining an internal temperature of 25±5° C. The mixture was agitated at 25±5° C. for 60 min. to produce a cloudy bi-phasic mixture of D-Amphetamine free base. Deionized (DI) water (50.0 ml, 0.5 vol) is charged to the reactor over 5 minutes.

### Example 2

### Coupling of D-Amphetamine free with N,N′-Bis-Boc-L-Lysine(Boc)-OSu

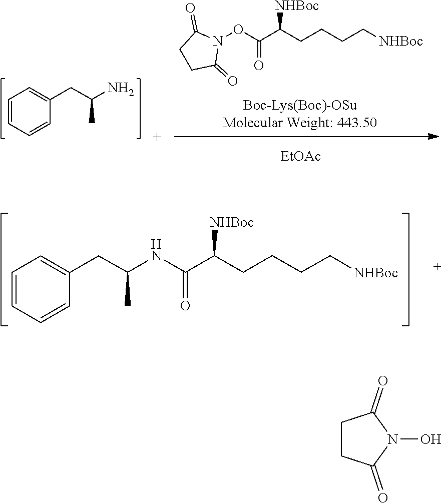

A separate 1 L glass jacketed reactor (Vessel 2) equipped with overhead agitation, temperature probe, nitrogen inlet, and addition funnel was charged with of N,N′-Bis-Boc-Lys(Boc)-OSu (1263 g, 2.1 eq.). Ethyl acetate (900 mL, 9 vol) was charged to Vessel 2 and heated until dissolution occurred. Dissolution of N,N′-Bis-Boc-Lys(Boc)-OSu occurred at 36° C. The solution of N,N′-Bis-Boc-Lys(Boc)-OSu in Vessel 2 was quickly filtered through a pre-warmed 250 mL fritted glass filter into a pre-warmed, 1 L 3-Neck round-bottomed flask (Vessel 3). Vessel 2 was rinsed with ethyl acetate (100 mL, 1 vol) and the rinse was transferred through the pre-warmed 250 mL fritted glass filter to vessel 3. Vessel 3 was maintained at temperature of 35° C. on a heating block attached to a hotplate. The solution of N,N′-Bis-Boc-Lys(Boc)-OSu in Vessel 3 was transferred, via cannula, to Vessel 1 over 60 minutes maintaining 25±5° C., to give a cloudy bi-phasic mixture that becomes clear over the course of reaction to produce N,N′-Bis-Boc-L-Lysine(Boc)-D-Amphetamine in the ethyl acetate portion/phase of the biphasic mixture and N-hydroxy succinimide (NHS) as a by-product in the aqueous portion/phase of the biphasic mixture. The reaction is agitated at 25±5° C. for 1.5 hrs. and the organic layer was sampled for completion.

### Example 3

### Removal of NHS from N,N′-Bis-Boc-L-Lysine(Boc)-D-Amphetamine

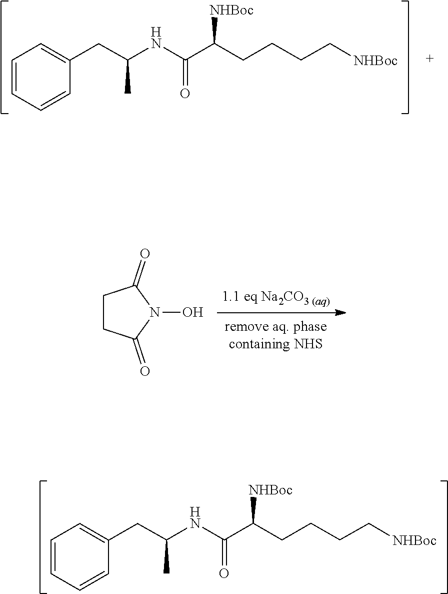

Aqueous sodium carbonate (31.64 g, 1.1 eq. in 500 mL, 5.0 vol water) was charged to the reactor over 15 min. using an addition funnel, while maintaining 20-25° C. After agitating for 15 min. at 20-25° C., agitation was stopped, the layers separated, and the bottom aqueous layer was removed. The organic layer was sampled for complete removal of N-hydroxysuccinimide (NHS).

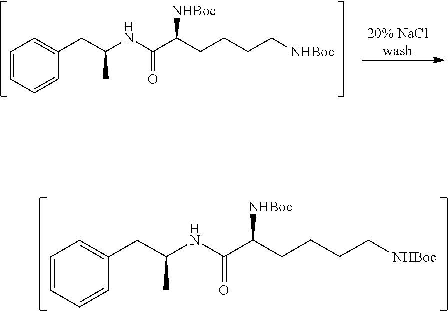

A 20% by weight aqueous solution of sodium chloride (200 mL, 2 vol) is charged to the reactor at 20-25° C. After agitating for 15 min. at 20-25° C., agitation is stopped, the layers were separated, and the bottom aqueous layer was removed.

### Example 4

### N,N′-Bis-Boc-L-Lysine(Boc)-D-Amphetamine: Solvent Replacement of Ethyl acetate for n-Propanol

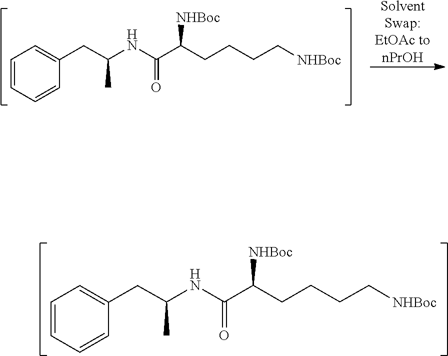

The ethyl acetate (organic phase) of N,N′-Bis-Boc-L-Lysine(Boc)-D-Amphetamine from Example 4 was concentrated by vacuum distillation to 1000 mL (10.0 vol), maintaining a temperature of 60° C. The solvent n-Propanol (1000 mL, 10 vol) was charged to the reactor, maintaining 40±5° C., and concentrated by vacuum distillation to 500 mL (10.0 vol), maintaining a temperature of 60° C. n-Propanol (1000 mL, 10 vol) was further charged to the reactor, maintaining 40±5° C., and concentrated by vacuum distillation to 1000 mL (10.0 vol), maintaining a temperature of 60° C. The mixture is sampled for residual ethyl acetate and water.

### Example 5

### Polish filtration of N,N′-Bis-Boc-L-Lysine(Boc)-D-Amphetamine

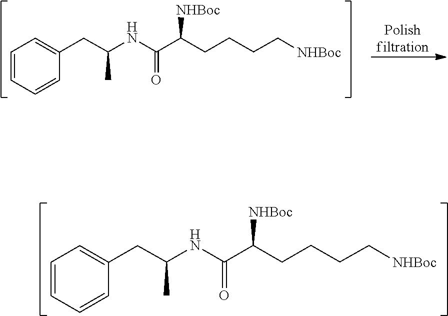

The n-propanol solution of N,N′-Bis-Boc-L-Lysine(Boc)-D-Amphetamine from Example 4 was polish filtered (0.2 μm) under vacuum via fritted funnel to remove inorganic salts.

### Example 6

### Addition of Methane Sulfonic Acid with N,N′-Bis-Boc-L-Lysine(Boc)-D-Amphetamine

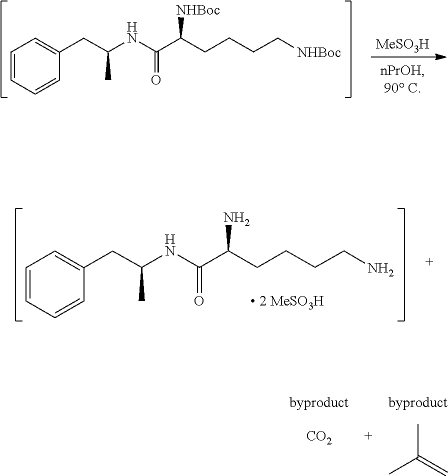

The polished filtered n-propanol solution of N,N′-Bis-Boc-L-Lysine(Boc)-D-Amphetamine from Example 5 was transferred, with propanol rinse (300 mL, 3 vol), to a clean 2 L jacketed reactor (temperature set to 40° C.) equipped with overhead agitation (PTFE, 4-blade pitched), temperature probe, condenser, nitrogen inlet, and addition funnel to give a clear solution. The solvent n-Propanol (700 mL, 7 vol) was charged to the reactor. The solution was sampled for purity. The solution was warmed to 90±3° C. (Tⱼ=97° C.). Methanesulfonic acid (260.8 g, 176.1 mL, 10.0 eq.) was added to the reaction dropwise via addition funnel (slightly exothermic) over 60 mins. Off-gassing of CO₂ and isobutylene was observed. The reaction was maintained at 90±3° C. for 60 min. to produce the API L-lysine-D-amphetamine dimesylate and was sampled for completion.

### Example 7

### Seed Crystallization of API L-lysine-D-amphetamine dimesylate

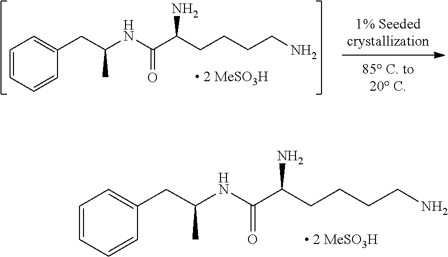

The reaction from Example 6 was cooled to 85° C. and stirred 10 minutes. Crystalline Lisdexamfetamine dimesylate (12.50 g, 1 wt %) was added as seed for crystallization. If the seed sustained crystallization of the API, the mixture was stirred for one hour, then cooled as described in step 23.

If the seed did not sustain crystallization, the mixture was cooled to 80° C. and stirred for 10 minutes. Lisdexamfetamine dimesylate (2.50 g, 1 wt. %) was further added as seed for crystallization. The mixture was cooled and seeded in 5° C. increments until the seed sustained crystallization. The mixture was then cooled as described. The slurry is cooled to 60±2° C. over 10 hours, then to 20° C. over 4 hours. The slurry of API was stirred at 20° C. for 8 hours. The slurry was filtered (fast filtration), washed twice with n-propanol (2×300 mL, 2×3 vol), pulled dry under vacuum. During vacuum drying, a nitrogen stream was maintained due to the hygroscopicity of the solid. Yield: 226.3 g, 91.5%

1. A process for preparing L-lysine-D-amphetamine dimesylate comprising the steps of: (a) reacting D-amphetamine free base having the structure 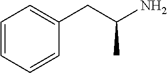
with about two equivalents of N,N′-bis-Boc-L-Lys(Boc)-OSu having the structure 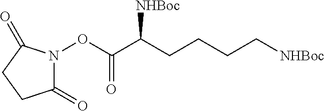 [SMILES: CC(C)(C)OC(=O)NCCCC[C@H](NC(=O)OC(C)(C)C)C(=O)ON1C(=O)CCC1=O]
in a biphasic mixture of ethyl acetate and water, heating the mixture from 25° to 35° C. with stirring to form N,N′-bis-Boc-L-lysine(Boc)-D-amphetamine having the structure 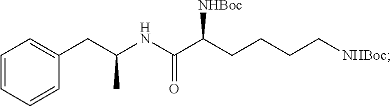 [SMILES: C[C@@H](Cc1ccccc1)NC(=O)[C@H](CCCCNC(=O)OC(C)(C)C)NC(=O)OC(C)(C)C]
and
(b) adding about 1 equivalent of sodium bicarbonate, then separating and removing the aqueous layer of the biphasic mixture and the replacing ethyl acetate portion of the biphasic mixture that includes the N,N′-bis-Boc-L-lysine(Boc)-D-amphetamine with n-propanol as a solvent, then adding about two equivalents to about 10 equivalents of methane sulfonic acid with heating up to 90° C. and stirring to result in the product L-lysine-D-amphetamine dimesylate.

2. The process of claim 1, wherein the reaction of step (a) is stirred from 1-2 hours, while heating the mixture from 25° to 35° C.

3. The process of claim 1, wherein in step (b) after the addition of sodium bicarbonate, the biphasic mixture is stirred up to 1 hour, maintaining a temperature for the stirred mixture of from 20° to 25° C.

4. The process of claim 3, wherein in step (b) after removing the aqueous layer of the biphasic mixture, the ethyl acetate portion of the biphasic mixture that includes the N,N′-bis-Boc-L-lysine(Boc)-D-amphetamine is further washed with an aqueous solution of sodium chloride and the biphasic mixture formed again is stirred up to 1 hour, maintaining a temperature for the stirred mixture of from 20° to 25° C., then the aqueous layer of the biphasic mixture formed is separated and removed prior to replacing ethyl acetate portion of the biphasic mixture that includes the N,N′-bis-Boc-L-lysine(Boc)-D-amphetamine with n-propanol as a solvent.

5. The process of claim 4, wherein in step (b) the N,N′-bis-Boc-L-lysine(Boc)-D-amphetamine in n-propanol undergoes polish filtration to remove any inorganic salts prior to addition of the methane sulfonic acid.

6. The process of claim 5, wherein in step (b) the about two to about ten equivalents of methane sulfonic acid is added to the N,N′-bis-Boc-L-lysine(Boc)-D-amphetamine in n-propanol solvent, heating the mixture from 85° to 90° C. with stirring for 1-2 hours to result in the product L-lysine-D-amphetamine dimesylate.

7. The process of claim 1, further comprising a step of: (c) adding a 1 percent by weight seed of L-lysine-D-amphetamine dimesylate while cooling the mixture of step (b) to aid in crystallizing the L-lysine-D-amphetamine dimesylate product.

8. The process of claim 7, further comprising washing the crystallized L-lysine-D-amphetamine dimesylate product with at least one portion of n-propanol solvent, followed by drying the crystallized L-lysine-D-amphetamine dimesylate product under vacuum at 50° C.

9. A process for preparing L-lysine-D-amphetamine dimesylate comprising the steps of: (a) reacting D-amphetamine free base having the structure 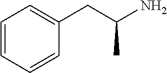
with about two equivalents of N,N′-bis-Boc-Lys(Boc)-OSu having the structure 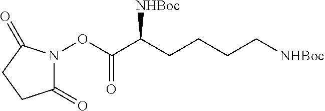 [SMILES: CC(C)(C)OC(=O)NCCCC[C@H](NC(=O)OC(C)(C)C)C(=O)ON1C(=O)CCC1=O]
in a biphasic mixture of ethyl acetate and water, heating the mixture from 25° to 35° C. for 1-2 hours with stirring to form N,N′-bis-Boc-L-lysine(Boc)-D-amphetamine having the structure 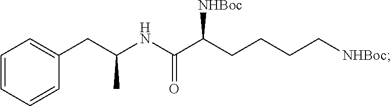 [SMILES: C[C@@H](Cc1ccccc1)NC(=O)[C@H](CCCCNC(=O)OC(C)(C)C)NC(=O)OC(C)(C)C]
(b) adding about 1 equivalent of sodium bicarbonate and stirring the biphasic mixture up to 1 hour, maintaining a temperature for the stirred mixture of from 20° to 25° C., then separating and removing the aqueous layer of the biphasic mixture;
(c) after removing the aqueous layer of the biphasic mixture in step (b), the ethyl acetate portion of the biphasic mixture that includes the N,N′-bis-Boc-L-lysine(Boc)-D-amphetamine is further washed with an aqueous solution of sodium chloride and the biphasic mixture formed again is stirred up to 1 hour, maintaining a temperature for the stirred mixture of from 20° to 25° C., then the aqueous layer of the biphasic mixture formed is separated and removed;
(d) after removing the aqueous layer of the biphasic mixture in step (c), replacing ethyl acetate portion of the biphasic mixture that includes the N,N′-bis-Boc-L-lysine(Boc)-D-amphetamine by concentrating the ethyl acetate under vacuum distillation and heating to 60° C. and adding n-propanol as a replacement solvent that includes the N,N′-bis-Boc-L-lysine(Boc)-D-amphetamine;
(e) polish filtering the N,N′-bis-Boc-L-lysine(Boc)-D-amphetamine in n-propanol solvent from step (d) to remove any remaining inorganic salts;
(f) adding about two to about ten equivalents of methane sulfonic acid to the N,N′-bis-Boc-L-lysine(Boc)-D-amphetamine in n-propanol solvent from step (e), heating the mixture from 85° to 90° C. with stirring for 1-2 hours to result in the product L-lysine-D-amphetamine dimesylate;
(g) cooling the L-lysine-D-amphetamine dimesylate product from step (f) and adding a 1 percent by weight of crystalline L-lysine-D-amphetamine dimesylate as a seed to crystallize the L-lysine-D-amphetamine dimesylate product; and
(h) washing the crystallized L-lysine-D-amphetamine dimesylate product with at least one portion of n-propanol solvent, followed by drying the crystallized L-lysine-D-amphetamine dimesylate product under vacuum at 50° C.
